(19) United States
(12) Patent Application Publication
(10) Pub. No.: US 2019/0328919 A1
(43) Pub. Date: Oct. 31, 2019

(54) PORTABLE DECOLONIZATION DEVICE

(71) Applicant: Salum Technologies LLC, Salt Lake City, UT (US)

(72) Inventors: Kristen M. Saad, Salt Lake City, UT (US); Azmi A. Ahmad, Salt Lake City, UT (US); Caitlynn Cooper, Orem, UT (US); Brooke Zhao, Mill Creek, UT (US)

(21) Appl. No.: 16/378,194

(22) Filed: Apr. 8, 2019

Related U.S. Application Data
(60) Provisional application No. 62/654,436, filed on Apr. 8, 2018.

(51) Int. Cl. A61L 2/24; A61L 2/10

(52) U.S. Cl. CPC A61L 2/24; A61L 2202/11; A61L 2202/16; A61L 2/10

(57) ABSTRACT

A portable decolonization device can include a casing and an ultraviolet light source housed within the casing. The ultraviolet light source can be adapted to produce an emitted ultraviolet light. A filter can be oriented to filter the emitted ultraviolet light to produce a filtered ultraviolet light having a center wavelength and a narrowed bandwidth, wherein the narrowed bandwidth is from 150 nm to less than 230 nm. An aperture in the casing can be oriented to allow at least one of the filtered ultraviolet light and the emitted ultraviolet light to pass out of the casing.

### RELATED APPLICATION

This application claims the benefit of U.S. Provisional Application No. 62/654,436, filed Apr. 8, 2018, which is incorporated here by reference.

### BACKGROUND

Bacteria is normal part of the human existence. Even the existence of methicillin-resistant Staphylococcus aureus (“MRSA”) is part of the normal human flora. However, for immuno-compromised patients it can cause devastating infections. To prevent these infections, a common treatment method is to use antibiotics. But, the use of antibiotics leads to further problems as it does not kill all the bacteria and results in the next generation becoming more resistant to antibiotics. Another problem with the use of antibiotics in treatment is that before prescription, hospital staff needs to take a sample and culture it to know if the patient needs the ointment or pills. This is a process that requires multiple days and visits, costing the hospital further time and resources.

### SUMMARY

A portable decolonization device can include a casing and an ultraviolet light source housed within the casing. The ultraviolet light source can be adapted to produce an emitted ultraviolet light. A filter can be oriented to filter the emitted ultraviolet light to produce a filtered ultraviolet light having a center wavelength and a narrowed bandwidth, wherein the narrowed bandwidth is from 150 nm to less than 230 nm. An aperture in the casing can be oriented to allow at least one of the filtered ultraviolet light and the emitted ultraviolet light to pass out of the casing.

In some cases, the casing further includes a head cap including the aperture. The head cap can allow for assembly of the device, access for bulb replacement, or otherwise facilitate repair or cleaning of the device.

Generally, the casing includes only a single opening such that ultraviolet light only leaves the casing through the filter. In some cases, the filter can be oriented within the housing, an associated head cap, or other portion of the device casing. Alternatively, a filter may be oriented within a tip which is secured within the aperture. For example, the tip can be a tube or other conduit through which ultraviolet light is directed. In this manner, unfiltered ultraviolet light does not leave the device so as to prevent undesirable exposure of unfiltered ultraviolet light to tissue or a patient.

The ultraviolet light source can be any suitable source of ultraviolet light which includes UV-C light having a wavelength spanning at least a portion of the range of about 150 nm to about 250 nm. In one specific example, the ultraviolet light source is a UV-C lamp. For example, UV-C lamps compliant with ISO 15858:2016 can be used. Non-limiting examples of UV-C lamps can include UV LED lights, UV mercury lamp, UV quartz lamp, low pressure UV lamp, other germicidal UV-C lamps, and the like. In one example, the UV-C lamp can be a UV-LED light formed of aluminum gallium nitride material. In some cases, the UV-C lamp can include a strip or collection of UV LED lights to obtain a desired light intensity. In another example, the ultraviolet light source can be a high-output germicidal UV-C lamp.

The ultraviolet light source can also be optionally housed within an ultraviolet reflective environment. For example, inner surfaces of the casing (i.e. outer or inner casings) can be lined with an ultraviolet reflective material. Non-limiting examples of ultraviolet reflective material can include expanded polytetrafluoroethylene, aluminum (e.g. aluminum sputtered on glass, aluminum foil, etc), 300 series stainless steel, and the like. A coating over a polished inner surface can keep the reflective material from oxidizing. Non-limiting examples of coating material can include silicon dioxide (SiO₂), Magnesium Fluoride (MgF₂), and the like. In one example, the ultraviolet reflective material can be a coated aluminum surface.

Although the device casing may serve as a lone housing element, in some cases it can be desirable to provide a removable unit (i.e. inner casing housing) which includes both the ultraviolet light source and the filter. The removable inner casing can also include electrical connectors or apertures for electrical connections to provide power to the ultraviolet light source.

The filter can be oriented at a location to filter the emitted ultraviolet light prior to exposure to a patient (i.e. release from the tip). Thus, although the filter may be oriented within the tip, most often the filter can be oriented within the casing. For example, the filter can be placed within a filter recess of the casing or head cap and adjacent the aperture.

The filter can generally be a UV bandpass filter. Optical bandpass filters are designed to transmit a well-defined wavelength of energy spectrum. The filter diameter can vary such as 12.5 mm, 25 mm, 50 mm, and the like. Surface quality of the bandpass filter can be 80-50 (Scratch-Dig) and lower, as specified in MIL-PRF-13830B. Suitable band-pass filters can include any light filter which prevents passage of light outside of a narrowed bandwidth. In order to substantially reduce risk of tissue damage a range of 180 nm to 220 nm can be particularly useful since wavelengths of about 230 nm and above have shown some elevated risk to viable tissue, depending on intensity and duration of exposure. In one example, the center frequency is within 15 nm of 202.79 nm or a filter of 202 nm with a full-width half-maximum (FWHM) of 15 nm. In another example, a 200 nm bandpass filter with a 10 nm FWHM can be used. Generally, a FWHM from 5 to 15 nm can be useful.

In some cases, the ultraviolet light source is a sole light source of the device, except an optional light source used for a display or indicator lights. In such cases, only a single light source is directed toward the target tissue.

In one alternative, the filter can be oriented within the aperture of the housing or casing. In this way the filtered light can be emitted from the device directly without using a tube or other light transfer element. Although a direct opening in the casing can be used to expose desired tissue, in many cases it can be desirable to have a tip which allows more flexibility in directing filtered ultraviolet light to target tissue (e.g. intranasally, intraorally, complex wounds, underneath adjacent tissue, etc). The tip can be secured within the aperture and have an inlet end and an outlet end. A light transfer element can be oriented within the tip so as to transfer the filtered ultraviolet light to the outlet end. For example, the light transfer element can be at least one of a UV reflective material (e.g. as discussed previously) coating inner surfaces of the tip and one or more optical fibers. In the case of optical fibers, inlet ends of such fibers can be associated with the filter so as to capture the filtered ultraviolet light. This can involve orienting the inlet ends within 1 mm (or within 0.5 mm) of the filter. A UV-fiber collimator can be used to convert divergent beams into a parallel beam when exiting the light source and passing through the filter. UV-grade fibers with a core diameter of 50 μm to 1000 μm can be used to prevent degradation of the UV light during transfer. Optical fibers can be used with a fiber coupler to combine multiple inputs into a single output, or split a single input into multiple outputs.

Exposure time and intensity can be controlled by varying power to the ultraviolet light source. Optionally, or in addition, a shutter can be oriented within the tip. Such a shutter can be electronically or manually actuated in order to variably block or allow filtered light to be emitted from the tip.

Further, a dispersion element can be oriented within the outlet end. A dispersion element can be any feature which disperses or broadens a light pattern of the emitted and filtered ultraviolet light so as to increase coverage of target tissue. Non-limiting examples of suitable dispersion elements can include a monochromator, a diffraction grating, a prism, a roughened reflective surface, or the like. Grating of the monochromator can be made from a master grating, where the surface has a number of parallel and closely spaced grooves in the range of 200-2000 grooves/mm.

Similarly, in some cases it can be desirable to shape emitted ultraviolet light in order to reduce treatment time, necessary movement, and/or more accurately target specific biological features. Accordingly, in some cases, the distal end can include a replaceable light shaping element. In one specific example, the replaceable light shaping element forms a light strip. Generally, the light strip has a length which is larger than a width (e.g. often from 15% to 400% larger). Non-limiting examples of light shaping elements can include a focused conical tip (FIG. 5) for use with intranasal or other focused area applications. Other examples include wide oval shaping elements (FIG. 6A-6C) which can be used over larger areas.

In order to effectively use the device, user input controls and output indicators can be used to convey operational information and allow a user to manipulate treatment. This in many cases, the device can further comprise one or more of a display screen and control buttons. A corresponding treatment control circuit can be adapted to vary one or more of exposure time, exposure intensity, and exposure patterns. These variables can be manually varied or can be controlled through pre-programmed treatment patterns. For example, a timer unit can be used to control exposure duration, and can be optionally programmable with preprogrammed times or preset times. Indicator lights or indicator display can often include a power indicator showing that the device is powered, and a UV indicator can show that UV light is being emitted from the device. Although exposure time may vary, most often the exposure time is from 20 seconds to 90 minutes, and in some cases from 5 minutes to 30 minutes. Similarly, the exposure dose can often vary from 10 to 500 mJ/cm², and in some cases from 100 to 250 mJ/cm². The dose can be dependent on a combination of factors including the wavelength, flux (watts), distance from source to surface, LED output patterns, and duration of exposure Regardless, the exposure intensity and duration can be sufficient to produce decolonization rates of equal or better rates than prescribed decolonization treatment (success rate of 39%). Surviving fraction of bacteria can be reduced to 4×10⁻³ orders of magnitude and higher.

In some cases, the exposure pattern is a multistage exposure including at least three exposure stages. Each stage can be varied in duration and intensity, or ramped. For example, an initial first stage can be 50% intensity, while a second stage can be 100% intensity. In some cases, the exposure pattern is a non-constant ramped exposure. In other cases, exposure can be a pulsed pattern. Non-limiting pulse pattern include pulses identical in UV-dose and separated by a specific time, pulses of varying doses separated by a specific time, or pulses of varying dose separated by a dose-dependent time.

A power source can be electrically connected to the ultraviolet light source. Such a power source can be a wall outlet, although in most cases it is desirable to have a portable device which is not externally wired. In such cases, the power source can be a rechargeable battery. Non-limiting examples of rechargeable batteries can include lithium ion battery or the like. In some cases, device is handheld such that the power source is directly structurally connected to the casing as an integrated unit.

In order to facilitate usability and storage, the casing can include a base which allows for stable vertical placement. For example, the base can include at least three coplanar feet or optionally can be formed as a substantially planar element.

Although specification may vary, operational temperatures can typically range from 60° F. to 100° F., while operating humidity tolerance can range from 0 to 100% humidity. Similarly, material of construction (e.g. casing, tip, and any components which may come in contact with patient tissue) can be biocompatible as defined by ISO 10993:2018, especially section 10.

Optionally, a wrist strap can be mechanically coupled to the casing in order to reduce accidental drop risks.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1A is a perspective view of a portable decolonization device according to one example.

FIG. 1B is a cross-section view of the device in FIG. 1A taken across B-B.

FIG. 2A is a side partial dissembled view of the device of FIG. 1A.

FIG. 2B is a cross-section view of the view of FIG. 2A taken across A-A.

FIG. 3 is a cross-section view of an inner casing of FIG. 1A.

FIG. 4A is a bottom perspective view of a head cap highlighting the filter recess in another example.

FIG. 4B is a side view of the head cap of FIG. 4A.

FIG. 4C is a cross-section view of the head cap of FIG. 4B taken across A-A and including a tip element in another example.

FIG. 5 is a side perspective view of a replaceable light shaping element as a focused conical tip for intranasal use in yet another example.

FIG. 6A is a top view of a head cap, tip and shaping element including a wide oval shaping element in still another example.

FIG. 6B is a side view of the tip assembly of FIG. 6A.

FIG. 6C is a bottom perspective view of the tip assembly of FIG. 6A.

FIG. 7 is a circuit diagram of one example control circuit.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

Definitions

In describing and claiming the present invention, the following terminology will be used.

The singular forms “a,” “an,” and “the” include plural referents unless the context clearly dictates otherwise. Thus, for example, reference to “a button” includes reference to one or more of such features and reference to “subjecting” refers to one or more such steps.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, “center wavelength” refers to the geometric mean of a lower cutoff wavelength and an upper cutoff wavelength of the filter where the maximum value of light is transmitted, and following a bell curve decay till the cutoff values (e.g. bandpass filter).

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

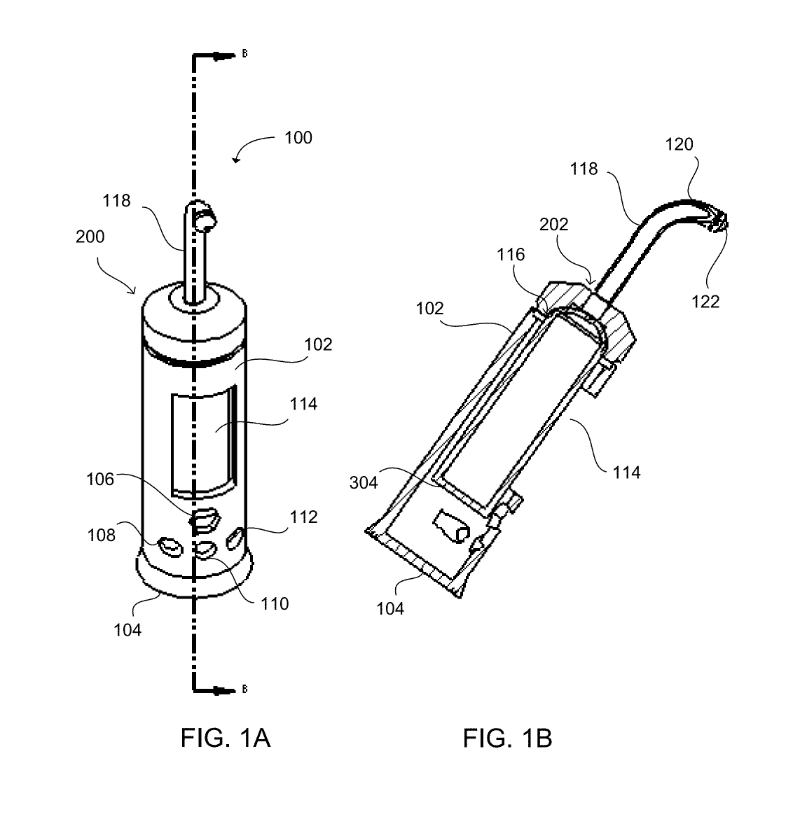

The present invention relates to a handheld decolonization device. More specifically, as shown in FIG. 1A, one embodiment comprises a casing 102 formed of a rigid material that is hollow on the inside with an opening 116 at the top of the casing 102. A base 104 may be placed at the bottom of the casing 102. A port may be placed in the bottom of the base 104 to allow the embodiment to be connected to a power supply 602. For example, in one embodiment, the device may be powered by a battery. Thus, the port may allow a charging cable to be connected to the device. In yet another example, the device may be powered through a wired connection. Therefore, the port may allow the device to be connected to the power cable.

One or more openings may be placed in the casing 102 to provide human interface elements. Specifically, referring to FIG. 1A, a power opening 106 may be located in the casing as a location for a power switch. Magnitude opening 108 may be located in the casing near the power opening 106. The magnitude opening 108 may provide for a control dial or button to allow the user to adjust the magnitude of light to be emitted. A notification opening 110 may be placed below the power opening 106 and to one side of the magnitude opening 108. The notification opening 110 may allow for an alarm light which indicates whether or not filtered ultraviolet light is being emitted. A timer opening 112 may be formed in the casing, to sit near the notification opening 110. The timer opening 112, may be used to place an interface device that may be used with a timer. For example, in one embodiment a dial may be used as a timer. In addition to the openings for human inputs, a screen opening 114 may be formed in the wall of the casing 102. A screen may be used to output information to the user, such as the power level, treatment profiles, elapsed time, error codes, or the like. The casing 102 may have an external attachment or handle to allow for stable carrying of casing. For example, rubberized portions can be molded into the casing to improve tactile grip and comfort.

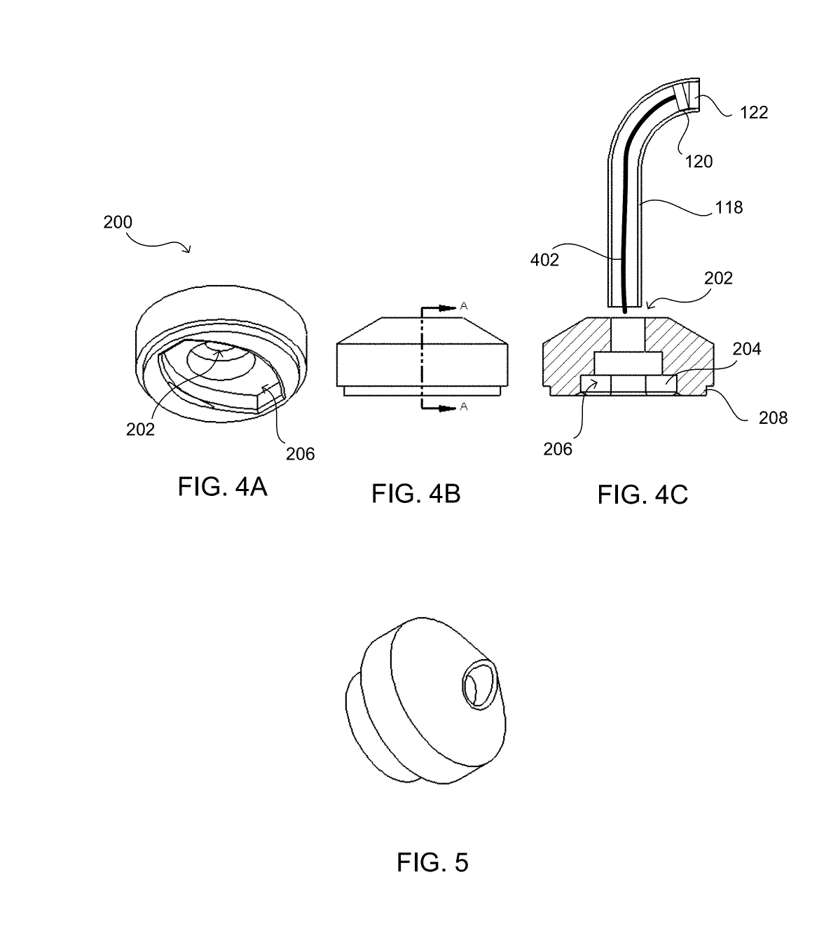

FIGS. 4A, 4B, and 4C show one embodiment of the head cap 200. The head cap 200 may be formed of similar material as the casing. Thus, the head cap 200 may be formed of either a UV reflective material or another rigid material that has been coated on the inside with a UV reflective coating. An aperture 202 may be formed in the top of the head cap 200. The aperture 202 may pass through the head cap and to the filter 204. The filter 204 may be placed with the head cap 200. As previously discussed, in one embodiment, the light source 302 may emit UV light at approximately 254 nanometers. However, this frequency of UV light may be dangerous to humans, thus, the filter 204 may be used to filter the light a safer 207 nanometer wavelength. It should be appreciated that this discussion pertains to only one embodiment and should not be construed to limit the device to only these wavelengths. An exemplary embodiment of the filter 204 may be a band-pass UV filter with a center wavelength of 202.79 nanometers. However, the mention of this center wavelength should not be narrowly construed. Center wavelengths between 185 nanometers and 225 nanometers should allow for decolonization without damage to human cells.

FIG. 4C shows where the head cap 200 may have an aperture 202 and the filter 204. In this embodiment the head cap 200 may have an additional space for a filter mount 206. However, it should be appreciated, that the filter mount 206 should not be limited to the shape shown in FIG. 4A and 4C. Rather, these views shows only an exemplary embodiment. In yet another embodiment, a collimator may be included in the head cap to narrow the light flow from the light source 302 and direct through the filter 204 and the head cap 200 out the aperture 202 to the light transfer tip 118.

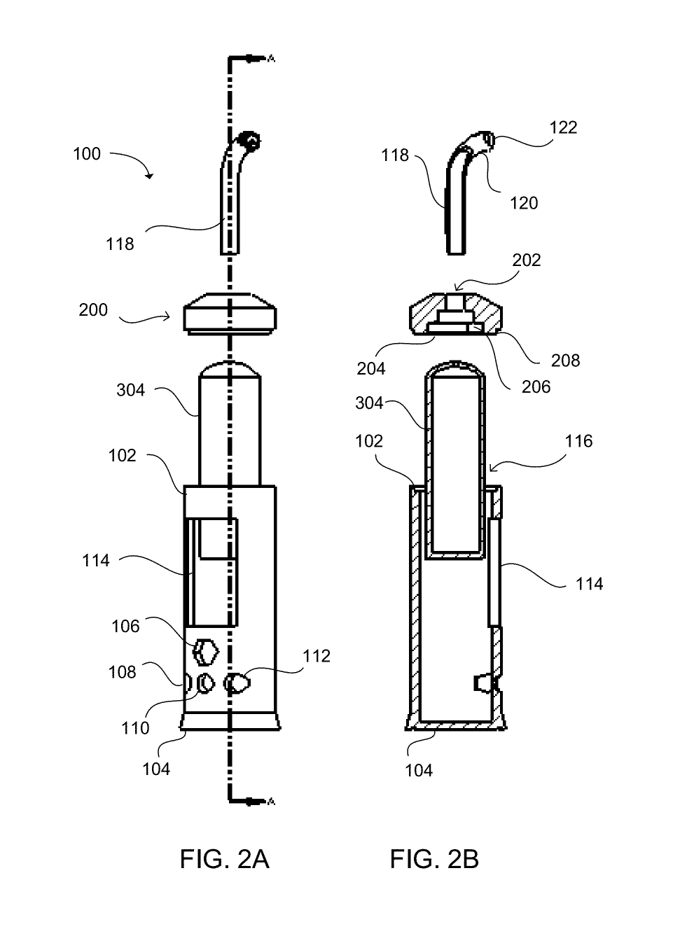

FIG. 4B embodiment further shows one method through which the head cap 200 and the casing 102 may be connected. Specifically, FIG. 4C shows a lip 208 may be nested within the opening 116 (FIG. 2B). In another embodiment, the head cap 200 may engage with the opening 116 of the casing 102 through a threaded engagement. The method to connect the head cap 200 and the casing 102 may be connected via any method that may function to prevent UV light from leaking out through the connection. The head cap 200 and the casing 102 may be produced as one piece, with an additional opening (similar to 114 but on the back) to allow access to internal light casing.

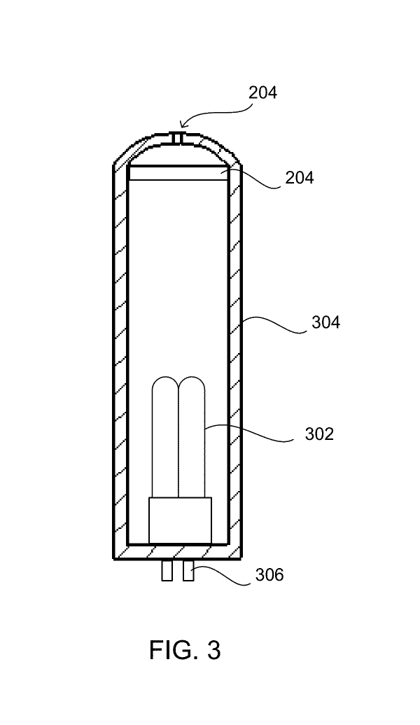

FIG. 3 shows one embodiment where a light source 302 may be placed within the inner casing 304. The light source 302 may be positioned such that the light emitted may pass through the filter opening 204 at the top of the inner casing 304. In one embodiment, the light source 302 may be an ultraviolet (“UV”) lamp that may emit UV light with a wavelength of 254 nm. In another embodiment, the light source may be a custom built UVC diode (LED), emitting UV light at a central wavelength of 210 nm. However, it should be appreciated that the description of this embodiment should not limit the scope of this device. Rather, these in two possible light sources. It should be appreciated that any other light source that emits light that may be used, either filtered or unfiltered, to decolonize falls within the scope of this invention. In another embodiment, an inner casing 304 may hold the light source 302. The inner casing 304 may be placed within and removed from the casing 102. Thus, in this embodiment an additional opening may be necessary to allow the user to insert and remove the inner casing 304 from the casing 102. In these embodiments, the casing 102 and the inner casing 304 may be formed of a UV reflective, yet rigid, material. Or, the casing 102 and the inner casing 304 may be formed of a rigid material and then the interior of both the casing 102 and the inner casing 304 may be coated with a UV reflective coating. Electrical connectors 306 can be used to transmit power to the light source.

Returning to FIG. 1A and 1B, a tip 118 may be engaged with the aperture 202 at the top of the head cap 200. As with the head cap 200, the tip may be inserted into the aperture as one method for preventing UV light from leaking out of the connection. As discussed earlier, many different connection methods may be used to connect the tip 118 to the aperture 202 to make sure the tip 118 is securely placed on top of filter 204 and inner casing 304 slot (see FIG. 2B). In another embodiment, the tip 118 may be connected to the aperture 202 through a threaded connection. The tip 118 may come in a variety of shapes, the tip 118 shown in FIGS. 1A through 2B is just one embodiment. In another embodiment the tip 118 can be a straight shape without a bend in it.

As shown in FIG. 4C, the transfer element 402 may be configured to transfer the light from the light source 302. In one embodiment, the transfer element 402 may engage with the filter 204. The transfer element 402 serves as a conduit to absorb the light passing through the filter 204 and transferring the light through the tip 118 to a shutter 120 and a dispersion element 122. In one embodiment, the transfer element 402 may be fiber optic threads, however the transfer element should not be limited to only fiber optics threads. But, rather any medium through which the UV light may be transferred from the filter 204 to the shutter 120 and the dispersion element 122, such as a set of mirrors, UV reflective inner tip surfaces, and the like.

As shown in FIG. 4C, one of the purposes of the tip 118 is to provide a housing for a transfer element 402. The shutter 120 may be placed within the tip to prevent UV light from leaking out of the tip 118. Also, the dispersion element 122 may be located near the end of the tip 118. As the light is transferred to the dispersion element 122, it may be diffracted to allow for generalized exposure to allow for a broader decolonization. In one embodiment the dispersion element 122 may be a diffraction grating. However, other elements may be used to accomplish the goal of spreading out the UV light, such as using a coupler to split the input into multiple outputs set at a distance apart. In another embodiment of the tip, the dispersion element 122 is not necessary; such as when shutter is open, the light is dispersed evenly onto the desired surface.

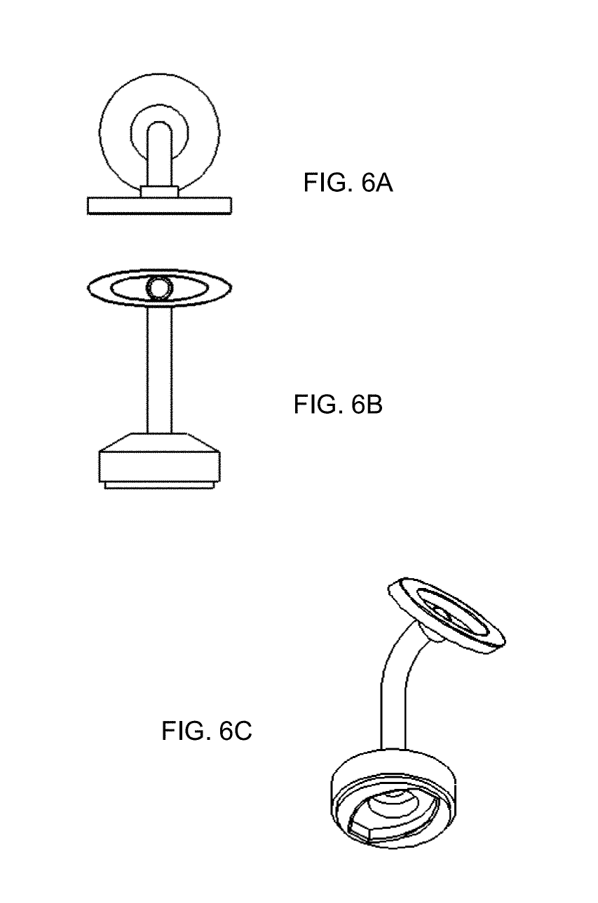

FIG. 5 shows one example of an intranasal replaceable light shaping element which is connectable to the end of the tip 118. In another embodiment, the tip 118 is not necessary; such as when the shutter 120 and dispersion element 122 can be placed inside the tip shown in FIG. 5, which is directly fitted onto the aperture 202. Many different connection methods can be used to connect FIG. 5 to the aperture 202. The tip can be nested into 202. In another embodiment, the tip can engage with the aperture 202 through a threaded connection. FIG. 6A through 6C illustrate an alternative replaceable light shaping element having a wide oval shape. Such shapes can be particularly suited to treatment of larger target tissue.

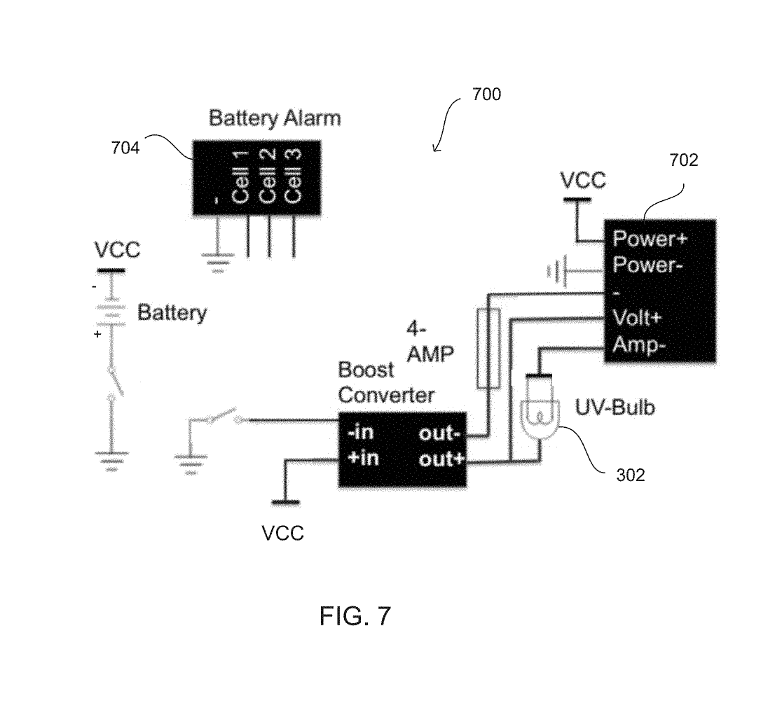

FIG. 7 shows an exemplary embodiment of the electronic circuit 700 that may be placed in the bottom of or within the casing. A power supply 702 may be placed on the circuit 700. In one embodiment the power supply may be a battery. However, in another embodiment the power supply may be a wired connection. If the embodiment uses a battery, then in another embodiment the battery may be placed within the casing. FIG. 7 further shows that a battery alarm 704 may be placed on the circuit 700. The battery alarm 704 may be connected with a light to indicate a low battery (e.g. through the notification opening 110 in the casing 102). The battery alarm 704 may also be used to set an alarm when too much battery is being drawn, or when the device is left ON for prolonged periods of time, even if the shutter is off. It should be appreciated, that FIG. 7 is simply an exemplary embodiment of the circuit 700, and that additional components may be used in order to achieve aforementioned or additional features. The various components of the present invention may be constructed generally out of any materials known to be suitable in the art.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A portable decolonization device, comprising:
a) a casing;
b) an ultraviolet light source housed within the casing and which is adapted to produce an emitted ultraviolet light;
c) a filter oriented to filter the emitted ultraviolet light to produce a filtered ultraviolet light having a center wavelength and a narrowed bandwidth, wherein the narrowed bandwidth is from 150 nm to less than 230 nm; and
d) an aperture in the casing oriented to allow at least one of the filtered ultraviolet light and the emitted ultraviolet light to pass out of the casing.

2. The device of claim 1, wherein the casing further includes a head cap including the aperture.

3. The device of claim 1, wherein the casing includes only a single opening such that ultraviolet light only leaves the casing through the filter.

4. The device of claim 1, wherein the ultraviolet light source is a UV-C lamp.

5. The device of claim 1, further comprising a removable inner casing housing both the ultraviolet light source and the filter.

6. The device of claim 1, wherein the filter is oriented within the casing.

7. The device of claim 1, wherein the filter is a UV bandpass filter.

8. The device of claim 1, wherein the center wavelength is within 15 nm of 202.79 nm.

9. The device of claim 1, wherein the ultraviolet light source is a sole light source of the device, except an optional light source used for a display or indicator lights.

10. The device of claim 1, further comprising a tip secured within the aperture and having an inlet end and an outlet end, and a light transfer element oriented within the tip so as to transfer the filtered ultraviolet light to the outlet end.

11. The device of claim 10, wherein the light transfer element is at least one of a UV reflective material coating inner surfaces of the tip and one or more optical fibers.

12. The device of claim 10, further comprising a shutter within the tip.

13. The device of claim 10, further comprising a dispersion element within the outlet end.

14. The device of claim 13, wherein the dispersion element is a diffraction grating.

15. The device of claim 1, wherein the distal end includes a replaceable light shaping element.

16. The device of claim 15, wherein the replaceable light shaping element forms a light strip.

17. The device of claim 1, further comprising one or more of a display screen and control buttons.

18. The device of claim 1, further comprising a treatment control circuit adapted to vary one or more of exposure time, exposure intensity, and exposure patterns.

19. The device of claim 15, wherein the exposure time is from 20 seconds to 90 minutes.

20. The device of claim 15, wherein the exposure dose is from 10 to 500 mJ/cm².

21. The device of claim 15, wherein the exposure pattern is a multistage exposure including at least three exposure stages.

22. The device of claim 15, wherein the exposure pattern is a non-constant ramped exposure.

23. The device of claim 1, further comprising a power source electrically connected to the ultraviolet light source.

24. The device of claim 16, wherein the power source is a rechargeable battery.

25. The device of claim 16, wherein the device is handheld such that the power source is directly structurally connected to the casing as an integrated unit.

26. The device of claim 1, wherein the casing includes a base which allows for stable vertical placement.
